(19) United States
(12) Patent Application Publication
(10) Pub. No.: US 2017/0360735 A1
(43) Pub. Date: Dec. 21, 2017

(54) INGESTIBLE EMULSION MATRIX FOR DELIVERY OF CREATINE

(71) Applicant: DreamPak, Mclean, VA (US)

(72) Inventor: Aly Gamay, Mclean, VA (US)

(73) Assignee: DreamPak, Mclean, VA (US)

(21) Appl. No.: 15/617,321

(22) Filed: Jun. 8, 2017

Related U.S. Application Data
(60) Provisional application No. 62/352,918, filed on Jun. 21, 2016.

(51) Int. Cl. A61K 31/198; A61K 47/26; A61K 9/16; A61K 9/107; A61K 9/00; A61K 47/44; A23L 33/175

(52) U.S. Cl. CPC A61K 31/198; A61K 47/44; A61K 47/26; A23L 33/175; A61K 9/107; A61K 9/0053; A61K 9/16

(57) ABSTRACT

Disclosed are compositions for improved oral delivery of creatine, methods of making such compositions, and kits comprising such compositions. In particular, the compositions are capable of dispensing a high dose of creatine in a convenient manner that does not require the consumption of liquids. The creatine is encapsulated in an emulsion matrix delivery system that contains a very low moisture level to keep the creatine in solid state, thus unexposed to moisture or acidic conditions, thereby preserving the creatine from hydrolysis, dissociation or other undesirable conversion into creatinine.

The emulsion matrix of the delivery systems provides for minimized degradation of the functional creatine during preparation of the matrix and the storage of the final delivery systems, thereby ensuring shelf stability. The use of relatively low temperatures in the preparation of the matrix, when compared to typical manufacturing procedures for other emulsions, ensures that the functional ingredients are not degraded by excessive heat.

### CROSS-REFERENCE TO RELATED APPLICATIONS

This application claims the benefit of U.S. Provisional Application No. 62/352,918, filed Jun. 21, 2016. The entire disclosure of the prior application is hereby incorporated by reference.

### STATEMENT REGARDING FEDERALLY SPONSORED RESEARCH OR DEVELOPMENT

This invention has been created without the sponsorship or funding of any federally sponsored research or development program.

### FIELD OF INVENTION

The present invention relates to creatine compositions for oral delivery, kits comprising such compositions, and methods of making such compositions.

### BACKGROUND

Oral dosage forms remain a significant problem for a significant segment of the population. Many individuals are unable or unwilling to swallow a solid dosage form. This problem occurs primarily in children and the elderly; however, problems with swallowing are not limited to those segments of the population. Certain conditions or disease states manifest themselves by swallowing difficulties. Otherwise healthy individuals can also exhibit problems with swallowing. Such swallowing difficulties irrespective of their cause can severely compromise patient compliance.

The nutraceutical industry has long-recognized the need for a form of oral administration, which avoids the swallowing difficulties associated with a traditional tablet. Syrups, elixirs, microcapsules containing slurries, chewable tablets and other tablet or capsule dosage forms have been developed; nevertheless, each has its own disadvantages. The disadvantages include a costly process for preparation and/or more costly packaging materials in addition to objectionable taste.

Current offerings of oral supplements in the market place include: dry pills or capsules (require a long time to dissolve in the stomach, has questionable absorption rate, may require several or large pills), dry powders (require mixing in large amounts of fluid making them inconvenient to carry or consume, have questionable taste), elixirs and syrups (bulky, hard to carry, and unpleasant taste have been deterrents to broad acceptance by consumers), chewable tablets and other chews (taste is questionable, smaller dosage than needed may be delivered). It is highly desirable to use portable monodose or single serve creatine supplements that can be consumed on the go.

Gel-like systems have been developed to deliver vitamins and other nutrients as confectionary products (gel confections). Exposing the gel systems to elevated temperature causes melt down in the gel structure and may cause separation of active ingredients among other phases. Melted gels could not be consumed as it is of runny consistency and become inconvenient to consume in particular at temperature above the melting point of the gel. Water may be added to the gel preparation in varying amounts, which, in turn, provides for higher moisture levels in the finished gel. Higher moisture may in turn impact both the efficacy of water sensitive compounds like creatine and shelf life of the finished gelled product. Furthermore, gelled systems require the addition of acidulants which in turn causes the creatine to deteriorate.

Creatine, also known as N-(aminoiminomethyl)-N-methylglycine, methylglycoamine, N-methyl-guanido acetic acid, or n-methyl-n-guanyl glycine, is widely distributed in tissues of the body, most notably in muscle, neural and reproductive tissues. Essentially, creatine is used biologically for the regeneration of ATP from ADP. Adenosine triphosphate (ATP) is the immediate source of energy for muscle contraction and neural activity. However, the amount of ATP in muscle fiber and neural tissue is relatively small and is utilized quickly during normal activity and even faster during exercise. Therefore, a backup supply of readily available energy to be used when ATP is diminished is a biological necessity. Creatine stored as phospho-creatine serves as a reservoir of high potential phosphoryl groups that are easily transferred to ADP by a phospho-kinase reaction to form ATP. In the process of regenerating ATP, phosphocreatine is used, and the creatine moiety of the molecule is spontaneously and irreversibly converted to its anhydride form, creatinine. Because creatine is irreversibly used, the body must either produce creatine biochemically or secure an outside source to supply the body with needed creatine. Creatine also is formed in the human body from two amino acids, glycine and arginine.

It is known that the oral ingestion of creatine will add to the whole body creatine pool, wherein the ingestion of 20-30 g creatine per day for several days can lead to a greater than 20% increase in the human skeletal muscle creatine content. Above a minimum plasma concentration, creatine enters the muscle fiber, accumulates and stays as phospho-creatine for several weeks. Thus, the strategy behind creatine supplementation is to consume the nutrient to capacity and then to take in only amounts sufficient to maintain full storage. This creatine loading phase dosage is estimated from the total creatine storage capacity of a person's body, which is, in turn, directly related to muscle mass, weight and exercise level. Recommended loading dosages, according to current literature values, are approximately 12-25 g/day. This daily dosage is usually divided into 3 or 4 doses per day. The maintenance dosages are determined using the same factors listed above and are approximately 4-15 g/day.

Although muscle tissue contains approximately 0.5% creatine by weight, the cooking process degrades most of the bioavailable creatine in meat. Furthermore, creatine is not well absorbed from the gastrointestinal (GI) tract, which has been estimated to have a 1-14% absorption rate. Thus, current products require large amounts of creatine to be administered to be effective, typically 5 grams or more. High levels of creatine dosing result in certain side effects. About 38% of men and 25% of women surveyed have indicated that they experienced serious side effects from consuming creatine. The most common complaints were diarrhea and flatulence. The incidence of side effects increases dramatically with large dosages (for example when greater than 12 grams is consumed) or by taking creatine on an empty stomach. Consuming large amounts of fluids with creatine may also contribute to the side effects. Athletes typically consume large amount of fluids to accommodate consumption of large portions of other necessary nutrients for optimal body building. It would be desirable to consume creatine in a concentrated form.

Furthermore, under acidic conditions, creatine is susceptible to cyclization and forms creatinine. In acidic aqueous solutions, the formation of creatinine from creatine is nearly quantitative and irreversible.

Nutritional and dietary supplements such as creatine have grown in popularity, as evidenced by the tremendous growth in the industry involved in their manufacture, production and distribution. Such creatine supplements can be consumed in a variety of ways, the most common being in powder or capsule form.

The consumption of creatine powders suffers from problems such as low suspension in water or liquids and unpleasant mouth feel and taste. Because of fast precipitation of creatine powder, the liquid has to be consumed immediately while being stirred. Nonetheless, creatine dose is often reduced due to loss in the serving container.

Many supplements are poorly absorbed into the body and a common approach to this problem is to consume larger doses, which can result in unpleasant side effects, including cramping, bloating and flatulence. Thus, more effective delivery systems are needed to improve oral delivery of creatine.

This background is provided for the purpose of making available information that is believed by the applicant to be of possible relevance to the present invention. No admission is necessarily intended, nor should be construed, that any of the preceding information constitutes prior art against the present invention.

### SUMMARY

An object of the present invention is to provide an oral delivery system for functional creatine. The present invention provides compositions and methods of delivering pharmaceutical doses of preserved creatine preparations in low water activity (Aw) emulsion matrices that are suitable for ingestion without the consumption of fluids or food. The compositions may be taken orally by humans or animals.

The present invention also provides methods of making of a composition for dispensing high doses of creatine neutraceutical preparations in a convenient manner that does not require the consumption of food or liquids. The methods include providing the neutraceutical preparations in emulsion delivery systems that contain low moisture levels to keep the active ingredient unexposed to moisture or low pH, thus preserving them from hydrolysis, dissociation or other undesirable reactions.

The present invention further provides methods for making a composition for dispensing high doses of creatine preparations in a soft emulsion matrix medium that has a low water activity (Aw) of about 0.70 or below to extend shelf life without adding preservatives and to improve stability against temperature fluctuations. The present invention further provides kits comprising a portable pre-formed package and a composition comprising an emulsion matrix and a creatine source component. The kits may be hermetically sealed. The hermetically sealed kits protect the composition and eliminate contamination and further packaging. The kits are portable and deliver the exact dose of actives that could be consumed directly without further addition to or mixing with liquids or solids.

The compositions, methods and kits of the present invention allow for high concentrations, not usually attainable in small serving sizes, of a creatine source component to be delivered orally at any temperature, whether low or elevated temperature (90°-140° F.), without melting or becoming runny.

It is an object of this invention to provide a low water activity (Aw) emulsion matrix that delivers certain neutraceutical preparations of creatine compounds and mixtures thereof.

Another object of this invention is to provide a predetermined water activity level of an emulsion matrix by controlling the amount of added water and incorporating humectants and other low water content liquids in order to protect creatine.

It is another object of this invention to provide an oral delivery system for creatine preparations that does not require the addition or ingestion of water and that preserves functionality and efficacy of creatine without the use of preservatives.

It is also an object of the present invention to provide a method for preparing a shelf stable oral delivery system that includes fat and emulsifiers to mask the objectionable taste of creatine compositions, to prevent clumping of creatine, and to provide a soft pasty consistency in order to facilitate dispensing of the compositions into packages and into the mouths of consumers with ease of consumption.

Other objects, features, benefits and advantages of the present invention will be apparent from the present disclosure to those skilled in the art, such as practitioners having knowledge of gelled products/compositions and their methods of preparation. Such objects, features, benefits and advantages will be apparent from the above as taken in conjunction with the accompanying examples, tables, data and all reasonable inferences to be drawn therefrom.

### BRIEF DESCRIPTION OF FIGURES

FIG. 1 is a graph showing the creatine and creatinine concentrations of an inventive composition as measured over a period of close to one year. The graph shows that creatine (5 grams/serving) was stable with virtually no conversion to creatinine throughout the accelerated shelf life study.

### DEFINITIONS

Unless defined otherwise, all technical and scientific terms used herein have the same meaning as commonly understood by one of ordinary skill in the art to which this invention belongs. Still, certain elements are defined below for the sake of clarity and ease of reference. These meanings are intended to supplement, rather than alter, the meanings of these terms as understood in the art.

“Shelf stable” means a food product that is microbiologically and chemically stable at ambient temperature without refrigeration or freezing.

“Low acid foods” means any foods other than alcoholic beverages, with a finished equilibrium pH greater than 4.6 and a water activity (Aw) value greater than 0.85. Tomatoes and tomato products having a finished equilibrium pH less than 4.7 are not classified as low-acid foods.

The term “pH” is used to designate the intensity or degree of acidity. The value of pH, the logarithm of the reciprocal of the hydrogen ion concentration in solution, may be determined by measuring the difference between the potential of two electrodes immersed in a sample solution.

The term “water activity level” is defined in the book “Food Science,” Third Edition, A.V.I. (1984), as a qualitative measure of unbound free water in a system that is available to support biological and chemical reactions. In general, as the water activity of a given food product decreases, its shelf life increases. A high water activity (Aw) product becomes more susceptible to mold, fungus and bacterial proliferation. For instance, the FDA defines a low acid food product with a pH of greater than 4.6 as shelf stable only if it has a water activity of 0.85 or less. Two foods with the same water content can vary significantly in their water activity depending on how much free water is in the system. When a food is in moisture equilibrium with its environment, the water activity of the food will be quantitatively equal to the relative humidity in the headspace of the container divided by 100.

The term “viscosity,” which is the resistance to flow, is mathematically defined as the shear stress divided by the rate of shear strain. “Shear stress” is the force acting in the plane of the fluid, and “shear rate” is the velocity gradient of the fluid between the plates. The shear rate takes into account the distance between the plates. It is defined in terms of the force required to move one plane surface continuously past another under specified steady-state conditions when the space between is filled by a specific liquid. Although absolute viscosity can be measured directly if accurate dimensions of the measuring instruments are known, it is more common to calibrate the instrument with a liquid of known viscosity (i.e., standard) and to determine the viscosity of the unknown fluid by comparison with that of the known. Foods exhibit different types of flow. In Newtonian materials, viscosity is not affected by changes in shear rate and remains constant. However, changes in shear rate do affect the viscosity of non-Newtonian materials.

The term “emulsion” means a mixture of two immiscible (unbendable) substances. One substance (the dispersed phase) is dispersed in the other (the continuous phase). Examples of emulsions include butter and mayonnaise. In butter, a continuous liquid phase surrounds droplets of water (water-in-oil emulsion). Emulsification is the process by which emulsions are prepared. Emulsions, being liquids, do not exhibit a static internal structure. The droplets dispersed in the liquid matrix (called the “dispersion medium”) are usually assumed to be statically distributed. Emulsions are part of a more general class of two-phase systems of matter called colloids. Although the terms colloid and emulsion are sometimes used interchangeably, emulsion tends to imply that both the dispersed and the continuous phase are liquid.

The term “lipid emulsifier source component” means a substance that stabilizes an emulsion by increasing its kinetic stability, such as a surfactant or surface active agent. Surfactants are compounds that are amphipathic, having a hydrophilic, water interactive “end,” referred to as their “head group,” and a lipophilic “end,” usually a long chain hydrocarbon fragment, referred to as their “tail.” They congregate at low energy surfaces, including the air-water interface (lowering surface tension) and the surfaces of the water-immiscible droplets found in emulsions (lowering interfacial tension). At these surfaces they naturally orient themselves with their head groups in water and their tails either sticking up and largely out of water (as at the air-water interface) or dissolved in the water-immiscible phase that the water is in contact with (e.g., as the emulsified oil droplet). In both of these configurations, the head groups strongly interact with water while the tails avoid all contact with water. Surfactant molecules also aggregate in water as micelles with their head groups sticking out and their tails bunched together. Micelles draw oily substances into their hydrophobic cores, explaining the basic action of soaps and detergents used for personal cleanliness and for laundering clothes. In one embodiment, the lipid emulsifier source component is selected from mono- and diglycerides, distilled monoglycerides, glycerol mono-stearates, sorbitan esters of hexitol anhydrides, sucrose esters, polyoxyethylene sorbitan esters of hexitol anhydrides, and mixtures thereof. In another embodiment, the lipid emulsifier source component is selected from mono- and diglycerides.

“Creatine source component” means any source of creatine that is suitable for use in the compositions, methods and kits described herein. Non-limiting examples of creatine source components include creatine monohydrate and other creatine hydrates, creatine salts such as creatine citrate, creatine pyruvate, creatine phosphate and other salts, creatine chelates, pro-creatine compounds such as glycocyamine (guanidoacetic acid), and protected and modified forms, analogues, derivatives, optical isomers, biologically active salts and esters of creatine. In one embodiment, the creatine source component is selected from creatine monohydrate, creatine hydrates, creatine chelates, pro-creatine, isomers, salts or esters of creatine, and mixtures thereof. In another embodiment, the creatine source component is creatine monohydrate. In a further embodiment, the creatine source component is in a non-solubilized solid, dry state.

“Lipid source component” means any source of triglyceride that is suitable for use in the compositions, methods and kits described herein. Non-limiting examples of lipid source components include fats and oils (e.g., anhydrous milk fat, butter, cream, cocoa butter), partially or fully hydrogenated vegetable fats and oils (e.g., cottonseed oil, coconut oil, corn oil, soybean oil, peanut oil, sunflower oil, canola oil, palm kernel oil), tallow, lard, medium chain fatty acids and enzyme modified fat. In one embodiment, the lipid source component comprises one or more edible fat and/or oil selected from anhydrous milk fat, butter, cream, cocoa butter, partially or fully hydrogenated vegetable fats and oils, cottonseed oil, coconut oil, corn oil, soybean oil, peanut oil, sunflower oil, canola oil, palm kernel oil, tallow and lard, medium chain fatty acids and enzyme modified fat. In another embodiment, the lipid source component is soybean oil, canola oil or butter.

“Sugar source component” means any source of sugar that is suitable for use in the compositions, methods and kits disclosed herein. Non-limiting examples of sugar source components include glycerin (also known as glycerine or glycerol) and polyols (also referred to as sugar alcohols, polyhydric alcohols and polyalcohols), propylene glycol, lactitol monohydrate, erythritol, syrups of sweeteners such as maltose, fructose, glucose or other mono and disaccharides, and natural syrups such as honey, maple syrup and corn syrup. Non-limiting examples of polyols include maltitol, sorbitol, xylitol, mannitol, isomalt and hydrogenated starch hydrolysate. In some embodiments, the sugar source component has a minimal amount of water (about 30% by weight or less) and remains flowable at around 80° to 110° F. In other embodiments, the sugar source component is selected from sucrose, maltose, fructose, glucose, natural syrups, honey, polyols, maltitol, sorbitol, xylitol, mannitol, isomalt, hydrogenated starch hydrolysate, propylene glycol, maltitol monohydrate and erythritol. In further embodiments, the sugar source component is selected from polyols. In additional embodiments, the sugar source component is glycerin.

“Isomers” refer to different compounds having the same molecular formula. Non-limiting examples of isomers include stereoisomers. “Stereoisomers” refer to isomers that differ only in the way the atoms are arranged in space. “Optical isomers,” also known as “enantiomers,” refer to a pair of stereoisomers that are non-superimposable mirror images of each other. “Racemic mixture” refers to a 1:1 mixture of a pair of optical isomers or enantiomers.

“Such as” has the same meaning as “such as but not limited to.” Similarly, “include” has the same meaning as “include but not limited to,” while “including” has the same meaning as “including but not limited to.”

The singular forms “a,” “or,” and “the” include plural references unless the context dictates otherwise. Thus, for example, a reference to “a compound” may include one or more compound(s) and/or equivalent(s) thereof.

Any numerical range disclosed herein encompasses the upper and lower limits and each intervening value, unless otherwise specified.

Other than in the working examples, or where otherwise indicated, numerical values (such as numbers expressing quantities of ingredients, reaction conditions) as used in the specification and claims are modified by the term “about”. Accordingly, unless indicated to the contrary, such numbers are approximations that may vary depending upon the desired properties sought to be obtained. At the very least, and not as an attempt to limit the application of the doctrine of equivalents to the scope of the claims, each numerical parameter should be construed in light of the number of significant digits and ordinary rounding techniques.

While the numerical parameters setting forth the scope of the disclosed subject matter are approximations, the numerical values set forth in the working examples are reported as precisely as possible. Any numerical value, however, inherently contains certain errors necessarily resulting from the standard deviation found in its respective testing measurements.

### DETAILED DESCRIPTION

The present invention provides paste-like compositions that contain low moisture content (about 0-20% by weight) in a flowable form which allows for dispensing into final packages and easy consumption by consumers. Furthermore, the low moisture content allows for the incorporation of water sensitive creatine without adverse reactions and masks off-taste and off-flavors associated with creatine compounds when dissolved in water. The low temperature of creatine inclusion and low pH of emulsion preserve the nutrient in its original state without any physical change and without conversion to creatinine. The presence of lipids in the emulsion helps disperse creatine and provides smooth mouth feel upon consumption. Lipids also help calm the digestive system which can experience side effects as a result of consuming large doses of creatine.

Because the creatine source component used in the inventive compositions and methods exists in a non-solubilized solid state, the same state as creatine powder, it is not exposed to water's impact on the conversion of creatine to creatinine. Since the creatine source component is in dry state, acidity and other detrimental agents do not cause any deformation or loss of concentration. The dry state of the creatine source component incorporated into the emulsion colloidal matrix ensures a long stable shelf life.

The delivery systems according to the present invention are suitable for the administration of creatine source components to both human and animals in a convenient presentation.

The emulsion matrix of the present invention can be tailored for specific purposes. Thus, the delivery systems can be formulated with specific combinations of functional ingredients in order to produce specific physiological effects. For example, the delivery systems can be formulated with combinations of functional ingredients, for example, to promote sexual potency, promote endurance, promote cardiovascular health, control fat and/or cholesterol, promote healthy joints, maintain or improve bone density, enhance cellular anti-oxidant capacity, or control appetite in addition to delivering creatine derivatives. Other non-limiting examples include, delivery systems formulated with combinations of creatine and functional ingredients to promote energy, increase endurance, promote weight loss, promote muscle enhancement or improve digestion.

In some embodiments, the delivery systems of the present invention comprise one or more creatine source components substantially uniformly dispersed within an emulsion matrix. In further embodiments, the delivery systems comprise (1) a lipid source component, (2) a sugar source component, (3) a lipid emulsifier source component, and (4) a creatine source component. Flavorings, colorings, acidulates, buffers and/or sweeteners can be included in conventional amounts in the emulsified matrix in order to provide variation in flavor and appearance.

Due to the substantially uniform and complete dispersion of the functional ingredients within the emulsion matrix, the delivery systems are suitable for division into sub-units. For example, if a single unit of a delivery system of the invention is divided into several subunits, one subunit will contain the dose of the creatine and the others will include other ingredients or nutrients. Such division would allow for inclusion of non-compatible nutrients and ingredients and will prevent interaction between various components.

Creatine has low solubility in water. Increasing the temperature increases the solubility rate. For instance at 70° F., only about 1.4% creatine dissolves in water, while about 4.5% dissolves at 120° F. Lowering the pH of the solution also increases solubility. However, soluble creatine in water at lower pH suffers degradation of creatine to creatinine at a fast rate. Therefore, the current invention uses creatine at a solid state throughout the manufacturing process as well as the shelf life.

The emulsion matrix of the delivery systems provides for minimized degradation of the functional creatine during the preparation of the matrix and the storage of the final delivery systems, thereby ensuring shelf stability. The use of relatively low temperatures in the preparation of the matrix, when compared to typical manufacturing procedures for other emulsions, ensures that the functional ingredients are not degraded by excessive heat. In accordance with the present invention, the delivery systems may be prepared at a temperature of 150° F. or less. In one embodiment of the present invention, the delivery systems are prepared at or below a temperature of 125° F. In another embodiment, the delivery systems are prepared at or below a temperature of 100° F. In a further embodiment, the delivery systems are prepared at a temperature of about 60°-120° F. Low temperatures can be employed in the preparation of the delivery system because the emulsion matrix is formulated to remain flowable at temperatures at or about 50°-150° F.

In addition, the delivery systems maintain a low interaction with water during and after preparation of the matrix, which also contributes to the stability of the functional creatine dispersed therein. In accordance with the present invention, the final moisture content of the delivery systems is about 0-20%. In one embodiment, the final moisture content of the delivery systems is about 2-20%. In another embodiment, the final moisture content of the delivery systems is about 1-15%. In another embodiment, the final moisture content of the delivery systems is about 1-10%. In other embodiments, the moisture content is about 10-15% or about 15-20%, provided that the water activity (Aw) is maintained at about 0.7 or below.

In one embodiment of the invention, the water activity of the final delivery systems is below about 0.7. In another embodiment, the water activity of the final delivery systems is below about 0.6. In other embodiments, the water activity is below about 0.55, between about 0.45 and about 0.55, or between about 0.40 and about 0.55. Low water activity is essential for protecting creatine from degradation and provides protection against microbiological spoilage in low acid foods without the exposure to detrimental high heat treatment during processing and production.

In accordance with the present invention, degradation of the functional ingredients during the process of preparing the matrix is less than about 10%. In one embodiment, degradation of the functional ingredients during preparation of the matrix is less than about 1%. The emulsion matrix also provides for minimized degradation of the functional ingredients dispersed therein during storage of the final delivery systems under normal storage conditions. In accordance with the present invention, therefore, degradation of the functional ingredients during storage of the delivery systems under normal conditions is less than about 15%. In one embodiment, degradation of the functional ingredients during storage is less than about 1%.

The formation of the inventive colloidal emulsion matrix enhances the entrapment of creatine and the suspension of dry creatine, as well as coats or encapsulates the dry creatine particles.

The compositions of the present invention can be formulated such that its final pH is in the range of about 2.5 to about 8.5. In one embodiment, the inventive composition has a final pH of between about 5.0 and about 8.5. Acidic pH can promote degradation of creatine ingredients. The inventive composition is formulated to deliver creatine which is sensitive to, or reactive at, acidic pH; therefore, the final pH of the matrix is neutral to mildly basic. By neutral to mildly basic pH, it is meant that the final pH is between about 6.0 and about 8.5. In one embodiment of the present invention, the composition is formulated such that its final pH is between about 6.0 and about 7.5. In another embodiment, the final pH of the composition is between about 6.2 and 7.0.

In the final form, the emulsified delivery systems of the present invention have a paste like consistency and texture similar to spreadable peanut butter or sesame seed paste. The emulsion matrix of the delivery systems, therefore, is formulated to be of gooey texture at normal room temperature. In the event, however, that the emulsion matrix is exposure to elevated temperatures (120°-160° F.), the emulsion matrix is such that no phase or fat separation of the components occurs and it maintains its consistency and appearance. Unlike gel based and carbohydrate based delivery systems, the current emulsion matrix retains its format at a wide range of temperatures from about 50° to about 150° F. To the opposite of known gel based and carbohydrate based delivery systems, no phase or physical appearance changes are noticeable in the inventive emulsion matrix. While creatine in liquids can experience precipitation of creatine, the inventive emulsion matrix provides sustainable distribution of creatine without sedimentation, phase separation or physical structure alteration. Therefore, the finished product is suitable for consumption at any place at any time.

The present invention provides delivery vehicles for sport neutraceutical agents, by the formation of compositions for treating humans and animals. The compositions may be suitable for ingestion by toddlers, young children or the elderly without fear of suffocation.

All conditions characterized as having a need for ingestible, soft and non-chewable delivery methods can benefit from the compositions and delivery methods of the invention. Because of the soft and pliable texture of inventive compositions, they can be swallowed with no need for users to chew a composition or drink a liquid.

The inventive emulsion matrix allows for single delivery dosage forms to contain large doses of hydrophilic and lipophilic neutraceutical preparations. Lipophilicity, fat-liking, refers to the ability of a chemical compound to dissolve in fats, oils, lipids, and non-polar solvents. Thus, lipophilic substances tend to dissolve in other lipophilic substances. Lipophilicity, hydrophobicity and non-polarity (the latter as used to describe intermolecular interactions and not the separation of charge in dipoles) all essentially describe the same molecular attribute; the terms are often used interchangeably. Hydrophilicity refers to ability of a molecule to transiently bond with water through hydrogen bonds. A hydrophilic portion of a molecule is one that is typically charge-polarized and capable of hydrogen bonding, enabling it to dissolve more readily in water than in oil or other hydrophobic solvents. Hydrophilic and hydrophobic molecules are also known as polar molecules and non-polar molecules respectively.

The emulsion matrix essentially entraps creatine making it available for fast ingestion and delivery to the stomach without degradation, thereby enhancing absorption in the small intestine. The presence of the lipid source component in the emulsion matrix reduces side effects in the stomach and the amount of acid secreted, thus preserving more of the creatine for enhanced absorption. The lipid emulsifier source component promotes dispersion of the phase in which they do not dissolve very well. For example, proteins and amino acids dissolve better in water than in oil, so they tend to form oil-in-water emulsions (that is, they promote the dispersion of oil droplets throughout a continuous phase of water).

The viscosity of the inventive emulsion matrix is similar to that of spreadable peanut butter or tomato paste. In one embodiment, the viscosity of the inventive emulsion matrix is between about 30,000-250,000 centipoise. In another embodiment, the viscosity is about 50,000-100,000 centipoise. In a further embodiment, the viscosity is about 102,000-224,000 centipoise.

This viscosity of the inventive compositions is quite different from other creatine formats in the market place. For example, liquid creatine have significantly lower viscosity, while creatine gels have unmeasurable viscosity at room temperature due to the firm texture of the gels.

Without being bound to any theory, a multidimensional matrix might be configured by binding amino acids of creatine (glycine and arginine), lipid, emulsifiers and water in a net configuration that resists phase segregation and maintains the integrity of the matrix.

### Creatine Source Component

The present invention contemplates the use of creatine monohydrate and other creatine hydrates, creatine salts such as creatine citrate, creatine pyruvate, creatine phosphate and other suitable salts. Creatine chelates are also contemplated in the present invention. Pro-creatine compounds that metabolize to yield creatine in the body are also contemplated, such as glycocyamine (guanidoacetic acid), as are protected and modified forms of creatine that can be metabolized in the body. Finally, analogues, derivatives, optical isomers and biologically active salts or esters of creatine that provide the same pharmaceutical results are also contemplated.

The components (e.g., creatine source components, lipid source components, lipid emulsifier source components, sugar source components) of the inventive compositions are in any form that is systemically ingestible in an animal or human. The components may take on not only different forms, but also different consistencies and/or physical states. For example, they may be pre-hydrated, pre-solubilized, pre-coated, pre-encapsulated, microencapsulated, micronized, particulated, micro-particulated or prepared as timed-release components either individually or in various combinations.

It will be understood that the amount of the creatine source component to be included in the delivery system will be dependent upon the particular application for which the final product is prepared. In one embodiment of the present invention, the total amount of creatine in the delivery system is less than or equal to 60% by weight. In another embodiment, the total amount of creatine in the delivery system is less than or equal to 50% by weight. In another embodiment, the total amount of creatine in the delivery system is less than or equal to 30% by weight.

The inventive compositions and methods allow for the incorporation of high levels of creatine, as high as 60% by weight, because the emulsion matrix holds the creatine source component without any precipitation during processing, separation during handling, or melting at a wide range of temperature from about 40°-150° F. In one embodiment, the creatine source component is micronized to a particle size of about 200 mesh or less or about 20-100 microns. In another embodiment, the creatine source component has a particle size of about 40-50 microns. The formation of a stable emulsion helps the inventive compositions hold and deliver large amounts of creatine source components.

### Lipid Source Component

The lipid source components used herein include products that provide triglycerides, the physical characteristics of beverage concentrates and substantially fatty materials to whiten the concentrates. Fats and oils which may be used as lipid source components include anhydrous milk fat, butter, cream, cocoa butter, partially or fully hydrogenated vegetable fats and oils, such as, for example, cottonseed oil, coconut oil, corn oil, soybean oil, peanut oil, sunflower oil, canola oil, palm kernel oil, and the like, tallow, lard and mixtures thereof. Medium chain fatty acids or enzyme modified fat may also be utilized in the current invention.

The inclusion of lipid source components in the inventive emulsion compositions enhances the dispersion and delivery of fat soluble or fat immiscible components, thereby providing for improved taste perception of the finished emulsion products. The lipid source components can work as masking agents for bitter or astringent nutrients as well as carriers of added flavors.

The lipid source components aid in masking off-flavors and unpleasant taste and allow for the incorporation of fat soluble flavors and colors for enhancement of organoleptic properties. Furthermore, the fatty slippery structure of lipid source components enhances the finished products' flowability during packaging and consumption.

It was discovered unexpectedly that the lipid source components coat the creatine source components and prevent further reaction with water or other active ingredients in the emulsion matrix. The lipid source components also prevent the creatine source components from aggregating or imparting a hash, powdery mouth feel. In other words, the creatine source components are segregated by the emulsified lipid source components, which results in enhanced stability during storage and a creamy perception on the tongue. The lipid source components were found unexpectedly to exert a protective effect on the creatine source components by shielding them from harmful acidic compounds and low pH impact.

The particular amount of the lipid source component used in the present invention depends upon a variety of factors such as the desired textural properties in the finished product, total fat level and type, and strength of the emulsion matrix. Generally, however, good results are obtained when the total lipid source component is present at levels ranging from about 2%-20% by weight of the composition. Oils provide good media for blending and processing without any amount of water present in the system.

### Sugar Source Component

Suitable materials for use as sugar source components are those liquids and fluids with minimal amount of water (about 30% or less) and remain flowable at about 80°-110° F. Examples of such materials are glycerin (about less than 1% water, liquid at room temperature, viscous, stable, hygroscopic, clear, odorless, noncorrosive, and sweet tasting), and polyols (also referred to as sugar alcohols, part of polyols' chemical structure resembles sugar and part is similar to alcohols, the terms polyhydric alcohols and polyalcohols may also be used). Examples of polyols include without limitation maltitol, sorbitol, and xylitol, mannitol, isomalt and hydrogenated starch hydrolysate. Other examples of suitable sugar source components are propylene glycol, lactitol monohydrate, and erythritol. Other suitable sugar source components include syrups of sweeteners such as maltose, fructose, glucose or other mono and disaccharides, and natural syrups such as honey, maple syrup and corn syrup.

The sugar source component acts as a humectant in the inventive compositions and methods to reduce the water activity to the recommended level in addition to imparting a desirable taste. In one embodiment, the amount of sugar source component is about 5-35% by weight of the composition. In another embodiment, the amount is about 5-30% by weight of the composition.

The use of a sugar alcohol in place of carbohydrate sweeteners allows for both lowered water activity and lowered sweetness in addition to lowered viscosity. In one embodiment, the sugar alcohol is glycerin. In other embodiments, the sugar alcohol constitutes about 5-40%, about 5-28%, about 7-10%, or about 12% by the weight of the composition.

Sugar alcohols are usually incompletely absorbed into the blood stream from the small intestines which generally results in a smaller change in blood sugar than “regular” sugar sucrose. This property makes them popular sweeteners among diabetics and people on low carbohydrate diets. However, like many other incompletely digestible substances, over consumption of sugar alcohols can lead to bloating and diarrhea because they are not absorbed in the small intestine.

Without being limited to any mechanism of action, it is believed that the addition of glycerin allows for the elimination of pasteurization or other thermal processing, thus helping in attaining excellent aroma in the finished creatine product.

### Emulsifier Source Component

The compositions of the present invention may be prepared using external emulsifier source components. The emulsifier source components may be included in the inventive emulsion compositions to improve the ease of formation of the emulsion and to promote stability. Emulsifier source components which may be used include emulsifiers which are approved for use in foods, such as mono- and diglycerides, distilled monoglycerides, glycerol mono-stearates, sorbitan esters of hexitol anhydrides, sucrose esters, polyoxyethylene sorbitan esters of hexitol anhydrides, and combinations of such food approved emulsifiers. In another embodiment, the amount of emulsifier used varies in the range of about 0.05-1.5% by weight of the emulsion composition. In another embodiment, the amount of emulsifier is about 0.2-1.0% by weight of the composition.

In one embodiment, the emulsifier source component is a lipid emulsifier source component. The lipid emulsifier source components differ from salt emulsifiers which impact the pH of a product; thus, the lipid emulsifier source components enhance the buffering capacity, resulting in improved stability. The inventive compositions do not require phosphate or citrate salts or organic chemical emulsifiers which can cause textural or acidity modifications. Nonetheless, such agents may be added without harming the emulsion matrix of the present invention.

The lipid emulsifier source component creates a film over a lipid source component fat globule, thereby repelling any water in the system and allowing repulsive forces to keep the lipid source component suspended and in turn the creatine source component entrapped in the emulsion matrix.

### Optional Ingredients

Optionally, the emulsified matrix compositions of the present invention can include effective amounts of flavor agent(s) to provide desired flavors. Generally, flavoring agent(s) at about 0.01%-3% of the finished products are contemplated. Suitable non-nutritive sweeteners may also be used for sugar-free food products. Examples of non-nutritive sweeteners include sucralose, spartame, saccharin and other high potency sweeteners. Suitable materials for use as nutritive carbohydrate sweetening agents are well known in the art. Examples of sweetening agents include both monosaccharide and disaccharide sugars such as sucrose, invert sugar, dextrose, lactose, honey, maltose, fructose, maple syrup and corn syrup or corn syrup solids. Examples of nutritive carbohydrate sweetening agents include those selected from the group consisting of sucrose, glucose, fructose, and corn syrup solids.

The present compositions can optionally contain a variety of additional ingredients suitable for rendering such products more organoleptically acceptable, more nutritious and/or more storage stable. Such optional components may include coloring agents, preservatives, and acidity and pH modifiers (acids and alkaline) and mixtures thereof.

The present compositions and methods can further employ numerous types of vitamins, probiotics, enzymes, hormones, nutritional supplements, synthetic compounds or other nutritional compounds and mixtures thereof in various forms and shapes.

Examples of nutraceuticals that may be employed along with creatine in the inventive compositions and methods include without limitation: vitamins (e.g., A, B, C, D, E, K), minerals (e.g., iron, calcium, copper, zinc, chromium, potassium, phosphorus, magnesium), soluble and non-soluble fiber (e.g., pectin, oat bran, Psyllium, cellulose), probiotics (e.g., Acidophilus, Bifidobacterium), enzymes (e.g., proteinase, lipase), joint pain treatments (e.g., glucosamine, chondroitin, collagen, SAM-e, COX-2 compounds), thermogenic compounds, energy compounds (e.g., CoQ 10) sports nutrients (e.g., creatine and derivatives thereof and nitric oxide) and other sports and anabolic compounds, nutritional material (e.g., amino acids, L-glutamine, taurine, L-carnitine, whey proteins, animal and plant proteins, peptides), fatty acids (and derivatives thereof), and herbal preparations (e.g., ginseng, echinacea, goldenseal). The ingredients employed in the inventive compositions and methods may be of various forms, consistencies or physical states. The nutraceuticals that could be utilized in the inventive compositions and methods may be pre-hydrated, pre-solubilized, pre-coated, pre-encapsulated, microencapsulated, micronized, particulated, and micro-particulated or prepared as timed-release components either individually or in various combinations.

Turning to more of the details, lipid source components, sugar source components, emulsifier source components, creatine source components, water and other optional ingredients may be combined in a standard food mixing and processing vessel in any order of addition. No heating is required for blending. In one embodiment, the water activity (Aw) of the mix is brought to about 0.65 or below. In other embodiments, the water activity is brought to about 0.25-0.65 or about 0.50-0.60.

The method includes combining the above ingredients at a low temperature of about 50°-100° F., or around ambient temperature.

Any sequence of ingredients addition may be adopted before the incorporation of components. In one embodiment, water is added first to the processor. Next, the sugar source component is added with continuous agitation. Next, the lipid source component and emulsifier source component are added. The temperature of the system is maintained at about 70-115° F. before the addition of the creatine source component in order to minimize the detrimental impact of heat on the active ingredient. Neutraceutical preparations, flavors, sweeteners, acidity modifiers, colors or other optional ingredients are then added. The ingredients are blended until a homogenous emulsified configuration is obtained in order to distribute and separate the creatine source component particles, thus eliminating the harsh, sandy mouth feel associated with creatine products upon oral consumption. Furthermore, the blend is agitated until lipid coating of the creatine source component is achieved as determined by tasting the product and obtaining a smooth mouth feel.

In another embodiment, the lipid source component and the emulsifier source component are added first to a mixer. Next, the sugar source component is added with continuous agitation. The temperature of the system is maintained at about 50-75° F. before the addition of the creatine source component in order to minimize the detrimental impact of heat on the active ingredient. The ingredients are blended until a homogenous emulsified configuration is obtained in order to distribute and separate the creatine source component particles, thus eliminating the harsh, sandy mouth feel associated with creatine products upon oral consumption. Furthermore, the blend is agitated until lipid coating of the creatine source component is achieved as determined by tasting the product and obtaining a smooth mouth feel. Water activity measurement may be taken at this stage.

The resultant product has a flowable consistency suitable for further filling into containers.

In one embodiment, the emulsified creatine composition possesses a measured water activity (Aw) of about 0.50-0.59 and a pH of about 6.5-6.9. In another embodiment, the emulsified creatine composition has a viscosity of about 20,000-100,000 centipoise at about 75° F.

The emulsion matrix composition may be filled using any of the filling equipment known to those skilled in the art of packaging technology. For example, the emulsion matrix composition may be filled into plastic, glass, and synthetic material, paper or the like containers or packages.

The emulsion matrix composition may alternatively be dispensed into hermetically sealed packages for extended shelf life. Dispensing the compositions into hermetically sealed unit dosage forms offers portability, rigidity, and formability. It also provides protection against moisture, gas and microbiological contamination, thereby extending the shelf life of unit dosage form even after opening the main, exterior package.

The emulsified preparations may be handled and distributed at room temperature, refrigerated or frozen depending on the type of nutraceuticals compounds, distribution channels and end users.

### EXAMPLES

This invention is further illustrated by the following examples, which are to be regarded as illustrative only, and in no way limit the scope of the invention. The following non-limiting examples and data illustrate various aspects and features relating to the methods and products/compositions of this invention, including the surprising and unexpected modification, control and/or improvement of the water activity level through use/incorporation of humectants.

### Example 1

Creatine pastes were produced. The creatine emulsions were formulated using various levels of glycerin and water and processed at about 70° F.

The products were formulated as follows:

| | % | % | % | % |
|---|---|---|---|---|
| Ingredient | A | B | C | D |
| Glycerin | 20 | 25 | 30 | 35 |
| Soybean Oil | 12 | 12 | 12 | 12 |
| Creatine Monohydrate | 40 | 40 | 40 | 40 |
| Water | 28 | 23 | 18 | 13 |
| Total | 100 | 100 | 100 | 100 |

Resultant creatine emulsions were packaged in laminated foil pouches (10 grams each), sealed and stored at room temperature for about 30 days. The products contained on average a moisture content of about 13-28% by weight and water activity (Aw) of about 0.61 to 0.80.

After about 20 days of storage, phase separation was observed. Upon opening the foil pouch, oil gushed out and left a base of compacted creatine. The creatine was not flowable and was hard to get out of pouches.

### Example 2

Creatine emulsion pastes were produced according to the teachings of the present invention. The creatine emulsions were formulated using various levels of creatine, oil, glycerin and water and processed at about 75-85° F.

The products were formulated as follows:

| | % | % | % | % | % | % |
|---|---|---|---|---|---|---|
| Ingredient | A | B | C | D | E | F |
| Glycerin | 20 | 20 | 20 | 30 | 24 | 25 |
| Sugar | 0 | 0 | 0 | 5 | 0 | 0 |
| Soybean Oil | 12 | 12 | 12 | 10 | 15 | 5 |
| Mono and Di-Glycerides | 0.5 | 0 | 0.2 | 0.4 | 0.4 | 0 |
| Creatine Monohydrate | 40 | 40 | 45 | 50 | 60 | 60 |
| Sodium Citrate | 0 | 0 | 0 | 0.2 | 0 | 0 |
| Citric Acid | 0 | 0 | 0 | 0 | 0.1 | 0 |
| Flavor | 0 | 0 | 1 | 2 | 0.5 | 1.2 |
| Cocoa | 0 | 0 | 3 | 2.35 | 0 | 0 |
| Water | 27.5 | 28 | 18.8 | 0.05 | 0 | 8.8 |
| Total | 100 | 100 | 100 | 100 | 100 | 100 |

About 10 grams of the resultant functional nutrients were packaged in a multilayer polyester film, sealed and stored at ambient temperature as well as at 104° F. for 120 days. Emulsified creatine treatments were evaluated at 10, 20, 30, 60, 90 and 120 days.

The products had an average moisture content of about 2.2 to 28.4% and had a water activity (Aw) of about 0.33 to 0.71.

The organoleptic characteristics of the flavored creatine emulsions were desirable. No harsh, grainy or dandy mouth feel was observed. No phase separation or oiling off was observed at the two tested temperatures. Creatine remained suspended and the texture maintained a homogenous texture with a semi-solid consistency. Creatine did not pact or form a solid layer at the bottom of pouches. Ease of dispensing out of packages was noticed. The above surprising results were a product of adding a fat emulsifier to the blend of oil and creatine. Even in the absence of water, the surfactants produced unanticipated results in terms of keeping the integrity of the semi-soft paste and making it function as a spread.

### Example 3

Sports nutritional supplement was prepared according to the teachings of the current invention in order to illustrate that creatine monohydrate was stable without any conversion into creatinine over a time period of close to one year. Even exposure to a high temperature of 104° F. did not alter the stability of creatine as preserved by the emulsified matrix structure and no fat separation or powder precipitation was noticed.

The creatine emulsion was formulated as follows:

| Ingredient | % |
|---|---|
| Glycerin | 28.7 |
| Mono and Di-Glycerides | 0.7 |
| Canola Oil | 11 |
| Creatine Monohydrate | 46 |
| Flavor | 3 |
| Water | 10.6 |
| Total | 100 |

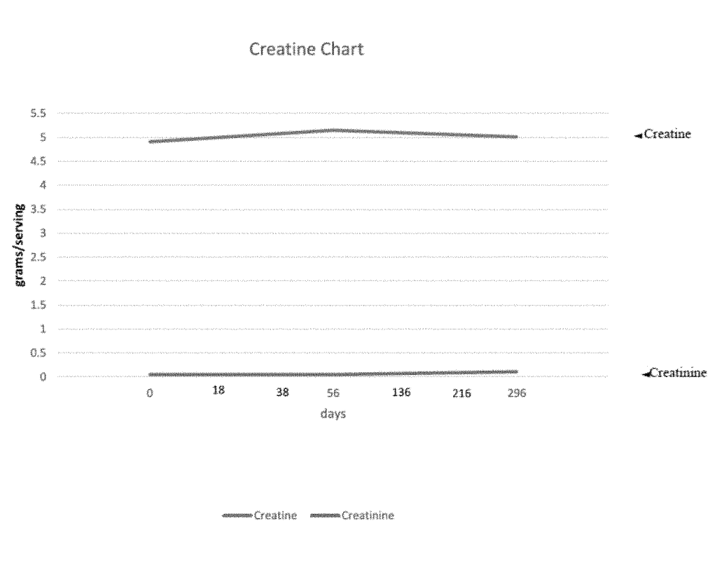

About 12.5 grams or the resultant creatine pastes were packaged in a multiplayer polyester film, sealed and stored at 104° F. for 120 days. Both creatine and creatinine concentrations were measured and graphed in FIG. 1.

The results illustrate that creatine (5 grams/serving) was extremely stable without any conversion to creatinine throughout the accelerated shelf life study. It was unexpectedly surprising to discover that the creatine, in a solid state suspended in the emulsion matrix, experienced no chemical reaction during storage at elevated temperature. Furthermore, the humectant tied up any free water in the media and virtually inhibited any chemical reaction to take place. It is expected that no creatinine presence will eventually be detected at any processing or handling temperature. The creatine in undissolved state was well preserved and will be delivered in its original whole configuration without chemical structure alteration.

### Example 4

To elucidate the incorporation of additional functional ingredients with creatine to enhance ease of consumption and functionality, the following creatine emulsions were formulated. Water and glycerin were mixed first in a mixing bowl at about 120° F. Creatine monohydrate was blended in, and oil and emulsifier were added followed by one of the actives, and mixed at 120° F.:

| | % | % | % |
|---|---|---|---|
| Ingredient | K | L | M |
| Glycerin | 30 | 35 | 40 |
| Mono and Di-Glycerides | 0.5 | 0.5 | 0.7 |
| Canola Oil | 8 | 10 | 15 |
| Creatine Monohydrate | 40 | 40 | 40 |
| L-Carnitine | 0 | 0 | 2 |
| Conjugated Linoleic Acid | 1 | 0 | 0 |
| Caffeine | 0 | 0.2 | 0 |
| Flavor | 5 | 5 | 1.5 |
| Water | 15.5 | 9.3 | 0.8 |
| Total | 100 | 100 | 100 |

The inventive creatine emulsions were convenient because of the portability of a stable semi-solid paste which could be carried to any place without fear of melting or phase separation. Consuming creatine by squeezing directly into mouth, without a need for fluid or utensils provided an excellent method to consume creatine products. The products possessed acceptable taste and flavor and generally exhibited excellent organoleptic characteristics.

### Example 5

Any form of creatine might be incorporated in emulsions. Furthermore, creatine derivatives employed in the method may be of various forms, consistencies or physical states and may be pre-hydrated, pre-solubilized, pre-coated, pre-encapsulated, microencapsulated, micronized, particulated, micro-particulated or prepared as timed-release components either individually or in various combinations as illustrated in the following inventive formulas.

Creatine forms were placed in a mixing bowl at about 70° F. Oil and emulsifier were added, followed by glycerin, sugar, butter, flavor and water. After thoroughly blending, the resulting products were filled into a multi-serve bottle.

| | % | % | % |
|---|---|---|---|
| Ingredient | O | P | Q |
| Glycerin | 10 | 30 | 40 |
| Sugar | 20 | 10 | 0 |
| Polysorbate 80 | 0.5 | 0.7 | 1.2 |
| Butter | 10 | 10 | 12 |
| Creatine Citrate | 40 | 0 | 20 |
| Encapsulated Creatine | 0 | 45 | 25 |
| Flavor | 1 | 1 | 1 |
| Water | 18.5 | 3.3 | 0.8 |
| Total | 100 | 100 | 100 |

Once the creatine paste was squeezed out of the bottle, it exhibited flowability and soft texture without precipitation of creatine compounds.

It will be apparent to those skilled in the art that specific embodiments of the disclosed subject matter may be directed to one or more of the above- and below-indicated embodiments in any combination.

While the principals of this invention have been described in connection with specific embodiments, it should be understood clearly that these descriptions, along with the chosen tables and data therein, are made only by way of example and are not intended to limit the scope of this invention, in any manner. Other advantages and features of this invention will become apparent from the following claims, with the scope thereof determined by the reasonable equivalents, as understood by those skilled in the art.

All references, publications, patents, and patent applications disclosed herein are hereby incorporated by reference in their entirety.

1. A composition comprising a creatine source component and an emulsion matrix, wherein the composition is formulated for oral delivery.

2. The composition of claim 1, wherein the creatine source component is selected from the group consisting of creatine monohydrate, creatine hydrates, creatine chelates, pro-creatine, isomers, salts or esters of creatine, and mixtures thereof.

3. The composition of claim 1, wherein the creatine source component is micronized to a particle size of about 10-100 microns.

4. The composition of claim 1, wherein the emulsion matrix comprises a lipid source component, a lipid emulsifier source component and a sugar source component.

5. The composition of claim 4, wherein the lipid source component comprises one or more edible fat and/or oil selected from the group consisting of anhydrous milk fat, butter, cream, cocoa butter, partially or fully hydrogenated vegetable fats and oils, cottonseed oil, coconut oil, corn oil, soybean oil, peanut oil, sunflower oil, canola oil, palm kernel oil, tallow, lard, medium chain fatty acids and enzyme modified fat; and the lipid source component is present at about 2-20% by weight of the composition.

6. The composition of claim 4, wherein the lipid emulsifier source component is selected from the group consisting of mono- and diglycerides, distilled monoglycerides, glycerol mono-stearates, sorbitan esters of hexitol anhydrides, sucrose esters, polyoxyethylene sorbitan esters of hexitol anhydrides, and mixtures thereof; and the lipid emulsifier source component is present at about 0.05-1.5% by weight of the composition.

7. The composition of claim 1, wherein the sugar source component is selected from the group consisting of sucrose, maltose, fructose, glucose, natural syrups, honey, polyols, maltitol, sorbitol, xylitol, mannitol, isomalt, hydrogenated starch hydrolysate, propylene glycol, maltitol monohydrate, and erythritol; and the sugar source component is present at about 5-40% by weight of the composition.

8. The composition according claim 1, further comprising a sweetener, a buffer, a natural or artificial flavoring, a coloring agent or a combination thereof.

9. The composition according to claim 1, further comprising one or more bioactive ingredients selected from the group consisting of botanicals, nutritional supplements, vitamins, minerals, enzymes, hormones, proteins, polypeptides, and mixtures thereof.

10. The composition of claim 1, wherein the creatine source component is substantially uniformly dispersed in said emulsion matrix.

11. The composition of claim 1, wherein said emulsified matrix is semi-solid at room temperature.

12. The composition of claim 1, wherein the emulsified matrix remains stable at a temperature of about 50°-150° F. without phase separation.

13. The composition of claim 1, wherein the composition has a moisture content of about 0-20% by weight.

14. The composition of claim 1, wherein the composition has a water activity of less than about 0.6.

15. The composition of claim 1, wherein the composition has a viscosity of about 25,000-250,000 centipoise.

16. The composition of claim 1, wherein the composition has a final pH of about 6.0-7.5.

17. A method of making an oral delivery composition comprising:
(a) blending one or more lipid source component, one or more sugar source component, one or more emulsifier source component, one or more creatine source component, and water at a temperature of about 60°-120° F. to prepare a blend;
(b) adjusting the moisture content of the blend such that the oral delivery composition has a moisture content of about 2%-20% and a water activity of less that about 0.7.

18. The method of claim 17, wherein the creatine source component is selected from the group consisting of creatine monohydrate, creatine hydrates, creatine chelates, pro-creatine, isomers, salts and esters of creatine, and mixtures thereof.

19-30. (canceled)

31. A kit comprising:
a composition comprising a creatine source component and an emulsion matrix, wherein the emulsion matrix comprises a lipid source component, a lipid emulsifier source component and a sugar source component; and
a preformed package.

32. The kit of claim 31, wherein the creatine source component is selected from the group consisting of creatine monohydrate, creatine hydrates, creatine chelates, pro-creatine, isomers, salts and esters of creatine, and mixtures thereof.

33-44. (canceled)
